(19) United States
(12) Patent Application Publication
(10) Pub. No.: US 2026/0335548 A1
(43) Pub. Date: Oct. 29, 2026

(54) DETERMINING ESTIMATED GLYCEMIC OUTCOMES FOR A USER OF A MEDICAMENT DELIVERY DEVICE BASED ON WEIGHTED SIMULATION SCENARIOS RESULTS

(71) Applicant: INSULET CORPORATION, Acton, MA (US)

(72) Inventors: Mert SEVIL, Manchester, NH (US); Anthony MEYER, Charlotte, NC (US); Joon Bok LEE, Acton, MA (US)

(21) Appl. No.: 19/656,531

(22) Filed: Apr. 23, 2026

Related U.S. Application Data
(60) Provisional application No. 63/793,140, filed on Apr. 23, 2025.

(51) Int. Cl. A61M 5/172; A61M 5/142; G16H 20/17

(52) U.S. Cl. CPC A61M 5/1723; G16H 20/17; A61M 2005/14292; A61M 2230/201

(57) ABSTRACT

The exemplary embodiments may provide an accurate prediction of a glycemic outcome for a user of a medicament delivery device, such as an insulin patch pump, when a change is made to the therapy provided by the medicament delivery device. The exemplary embodiments may provide a number of different simulation scenarios and may simulate these scenarios with current control method settings to yield predicted glycemic outcomes for each scenario. The exemplary embodiments may then seek to determine a weighted sum of the predicted glycemic outcomes of the scenarios that most closely matches an actual glycemic outcome as found in a glucose level history of the user. The weights and the in silico user associated with the chosen weighted sum are used in a simulation with a modified insulin therapy to produce a predicted glycemic outcome for the modified insulin therapy.

### CROSS-REFERENCE TO RELATED APPLICATION

This application claims the benefit of and priority to the U.S. Provisional Patent Application No. 63/793,140, filed on Apr. 23, 2025, and titled “DETERMINING ESTIMATED GLYCEMIC OUTCOMES FOR A USER OF A MEDICAMENT DELIVERY DEVICE BASED ON WEIGHTED SIMULATION SCENARIOS RESULTS,” the entire content of which is incorporated herein by reference in its entirety.

### BACKGROUND

Users of medicament delivery devices, such as automated insulin delivery (AID) devices, may wish to understand expected changes in outcomes with modified medicament delivery therapy. For users of AID devices, the users may wish to know how changes are expected to affect the glycemic outcomes (i.e., glucose levels of the users). For instance, suppose that there is an interest in seeing how changing a parameter of their insulin therapy (such as a change in a value of a control parameter) affects a glycemic outcome of a user. One conventional option is to perform a simulation per a scenario for a given in silico user. Unfortunately, such simulations do not produce accurate results. The simulated results of a glycemic outcome do not match the actual glycemic outcome of the user when the change in the parameter of insulin therapy is made.

### SUMMARY

In accordance with an inventive facet, a method is performed by one or more processors of a computing environment. Per this method, the one or more processors may determine a weighted mix of simulation scenarios of glycemic outcomes for a user of an insulin delivery device. The weighted mix may specify weights for results for each of the simulation scenarios. The method may further include the one or more processors determining an estimated glycemic outcome for the user given a change of at least one control system setting of the insulin delivery device by determining results of simulating with the one or more processors each of scenarios in the weighted mix of simulation scenarios with the changed at least one control system setting and weighing the results per weights specified in the weighted mix of simulation scenarios.

The determining of the weighted mix of simulation scenarios may be based on a glucose level history of the user. The weights may be determined by applying an optimization to yield weights that produce a weighted sum of scenario results that is similar to actual glucose levels of the user in the glucose level history of the user. The scenarios may include at least one of a scenario for high insulin sensitivity, a scenario for low insulin sensitivity, a scenario for the user fasting, or a scenario for the user exercising. The scenarios may include at least one scenario for pump occlusion, a scenario for pressure induced sensor error, a scenario for total daily insulin inaccuracy, or a scenario for glucose sensor error. The scenarios may include at least one scenario for high carbohydrate ingestion, a scenario for high fat ingestion, a scenario for ingesting multiple meals, or a scenario for eating meals closer in time than normal. Information may be output regarding a difference between the estimated glycemic outcome for the user given the change of the at least one control system setting versus an actual glycemic outcome for the user without the change of the at least one control system setting.

In accordance with another inventive facet, a non-transitory computer-readable storage medium may store computer programming instructions that when executed by a processor cause the processor to perform the method.

In accordance with a further inventive facet, a method is performed by a processor of a computing system. Per the method, the processor may simulate glycemic outcomes of different in silico users of an insulin delivery device for different scenarios using an initial set of control system settings. The in silico users may differ as to physiology. The processor may determine weights of the glycemic outcomes of the scenarios for each in silico user to produce a weighted sum of the glycemic outcomes for the scenarios that most closely matches an actual glycemic outcome for the user of the insulin delivery device given the initial set of control system settings. The processor may select a weighted sum of the glycemic outcomes for the scenarios for a selected one of the in silico users that most closely matches the actual glycemic outcome for the user of the insulin delivery device. The processor may simulate the scenarios for the selected one of the in silico users given a new set of system settings to determine an updated weighted sum of the glycemic outcomes for the scenarios for the selected in silico user and may output information regarding the updated weighted sum.

The method may include outputting information regarding the actual glycemic outcome for the user of the insulin delivery device given the initial set of control system settings. The determining of the weights may be based on glucose levels in a glucose level history of the user. The weights may be determined by applying an optimization to yield weights that produce a weighted sum of the glycemic outcomes for the scenarios that is similar to actual glucose levels of the user in the glucose level history of the user. The insulin delivery device may be an insulin patch pump. The method may include determining performance metric values for both the actual glycemic outcomes for the user of the insulin delivery device with the initial set of control system settings and the estimate of the glycemic outcome for the user of the insulin delivery device with the new set of system settings.

### BRIEF DESCRIPTION OF THE DRAWINGS

FIG. 1 depicts an illustrative medicament delivery system.

FIG. 2 depicts an illustrative computing system for exemplary embodiments.

FIG. 3 depicts an illustrative distributed computing environment for exemplary embodiments.

FIG. 4 depicts a flowchart of illustrative steps that may be performed in exemplary embodiments to estimate effects of changes in control method settings.

FIG. 5A depicts illustrative simulation scenarios that may be deployed in exemplary embodiments.

FIG. 5B depicts in silico scenarios in contrast with real life activities of a user.

FIG. 6 depicts a flowchart of illustrative steps that may be performed in exemplary embodiments to find a best in silico subject and weights.

FIG. 7 depicts a flowchart of illustrative steps that may be performed in exemplary embodiments to determine the best weights for each in silico subject.

FIG. 8 depicts a graphical representation of steps that may be performed in exemplary embodiments to determine the difference between the expected results of applying a change in control method settings and the results with current control method settings.

FIG. 9 depicts an illustrative table illustrating Time in Range (TIR) outcomes for selected simulation scenarios before and after a change in control method settings.

### DETAILED DESCRIPTION

The exemplary embodiments may provide an accurate prediction of a glycemic outcome for a user of a medicament delivery device, such as an insulin patch pump, when a change is made to the therapy provided by the medicament delivery device. The change may be a modification of one or more control parameter values and more generally entails at least one control method setting (also referred to as control system setting) change. The exemplary embodiments may provide a number of different simulation scenarios and may simulate these scenarios with current control method settings to yield predicted glycemic outcomes for each scenario. The term “simulate” as used herein refers to its common meaning in the art. In particular it may refer to computationally determining one or more predicted outcomes for a defined set of input conditions using a model or algorithm. The exemplary embodiments may then seek to determine a weighted sum of the predicted glycemic outcomes of the scenarios that most closely matches an actual glycemic outcome as found in a glucose level history of the user. The glycemic outcomes may be realized as glucose level values for a user over a time window. The optimal weights to match the actual glycemic outcome may be determined using an optimization method. The term “optimization method” as used herein refers to its common meaning in the art. In particular it may refer to any method configured to determine one or more values of a variable or parameter set that optimize an objective function, in particular minimize or maximize the objective function, optionally subject to one or more constraints. The simulation may be performed for a single in silico user (i.e., a single representation of the user for the simulation) or may be performed for multiple in silico users. Each in silico user may have a different physiology, such as a different weight, age, and/or sex. The optimal weights for the scenario glycemic outcomes are determined for each in silico user, and the weighted sum of the scenario glycemic outcomes for one of the in silico users with a smallest error relative to the actual glycemic outcome is chosen. The weighted sum is a glucose level value. The weights and the in silico user associated with the chosen weighted sum are used in a simulation with a modified insulin therapy to produce a predicted glycemic outcome for the modified insulin therapy. Output may be produced to enable a comparison of the predicted glycemic outcome for the modified insulin therapy with the actual glycemic outcome with the current control parameter values. Metrics may be applied to the glycemic outcomes to determine quality of glucose level control.

The approach described herein may be useful for clinical trials. For example, a clinical trial may be performed to determine how altering control method settings affects the glycemic outcomes of patients.

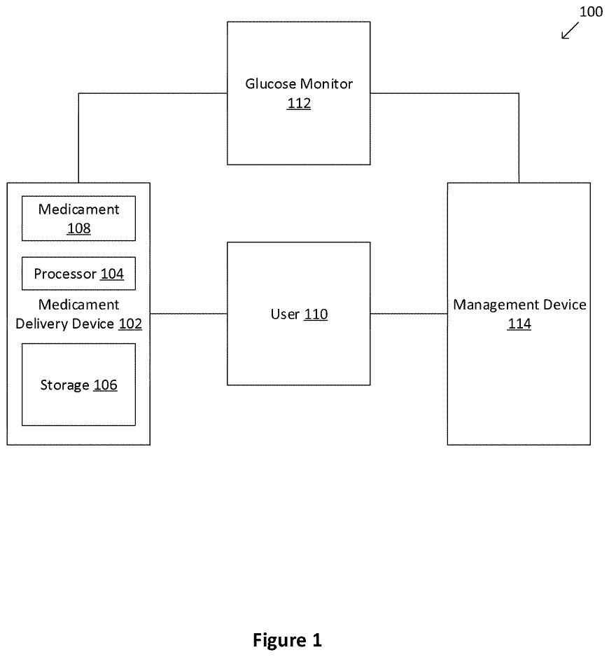

The methods described herein may be applied for a medicament delivery system 100, such as, for example, that depicted in FIG. 1. The medicament delivery system 100 may include a medicament delivery device 102 for delivering medicament to a user 110. The medicament delivery device 102 may be, for example, an on-body device like an insulin patch pump that is secured to the user 110 in some embodiments. In other embodiments, the medicament delivery device 102 may not be attached to the body of the user 110. The medicament delivery device 102 may include a processor 104. The processor 104 may be, for example, a microprocessor, a logic circuit, a field programmable gate array (FPGA), an application specific integrated circuit (ASIC) or a microcontroller. The processor 104 may be operable to execute a control application encoded in computer programming instructions stored in a storage 106 that enables the processor 104 to direct operation of the medicament delivery device 102 (i.e., to provide the control system). The control application may be a single program, multiple programs, modules, libraries or the like. A closed-loop control approach may be performed by the control application. The control application receives glucose level values from a glucose monitor 112 as input and may determine insulin delivery doses in response. The storage 106 may include both primary memory and secondary memory. The storage 106 may include random access memory (RAM), read only memory (ROM), optical storage, magnetic storage, removable storage media, solid state storage or other types of non-transitory computer-readable storage media. The storage 106 may store data, such as glucose level histories, insulin delivery histories and other data that is used in controlling insulin delivery to the user 110. The storage may also store computer programming code and other items, such as documents, files, etc.

It should be appreciated that the control application performs a control method for the medicament delivery device. The control method may include a number of settings that may affect the operation of the control method. Examples of control method settings include settings such as target glucose level, constraint values, correction factor, or the like.

The medicament delivery device 102 may include a medicament storage 108, such as a reservoir, a vial, or the like. The medicament stored therein may be insulin for lowering glucose levels of the user 110. The medicament delivery device 102 also may deliver a second medicament, such as glucagon, for raising glucose levels of the user 110. The medicament delivery device 102 may deliver a glucagon-like peptide (GLP)-1 receptor agonist medicament for lowering glucose or slowing gastric emptying, thereby delaying spikes in glucose after a meal. The medicament delivery device 102 may deliver a gastric inhibitory polypeptide (GIP) or a dual GIP-GLP receptor agonist. In other embodiments, the medicament delivery device 102 may deliver pramlintide, or other medicaments that may substitute for insulin. More generally, the medicament delivery device 102 may deliver a medicament for managing and/or affecting glucose levels of the user 110. In other embodiments, the medicament delivery device 102 may deliver concentrated insulin. In some embodiments, the medicament or medicaments delivered by the medicament delivery device may be a coformulation of two or more of those medicaments identified above. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agents, a nutritional supplement or a vitamin.

The medicament delivery system 100 may include a management device 114 for managing the medicament delivery device 102. The management device 114 may be a special purpose device, such as a dedicated personal diabetes manager (PDM) device. The management device 114 may be a programmed general-purpose device, such as any portable electronic device including, for example, a dedicated controller, such as a processor, a micro-controller, or the like. The management device 114 may be used to program or adjust operation of the medicament delivery device 102 and/or the glucose monitor 112. The management device 114 may be any portable electronic device including, for example, a dedicated device, a smartphone, a smartwatch, or a tablet.

It should be appreciated that other varieties of medicament delivery systems and medicament delivery devices than those that deliver insulin and have the components of FIG. 1 may be used. The depiction in FIG. 1 is intended to merely illustrative and not limiting. Moreover, the embodiments described in more detail below will focus on the case where the medicament delivery device 102 delivers insulin and the outcome of interest is a glycemic outcome. The exemplary embodiments may deliver different types of insulin as identified above and may measure analyte levels other than the glucose level. Other sensors values may be measured, such as, for instance, heart rate, respiration rate, blood pressure, galvanic skin response, etc.

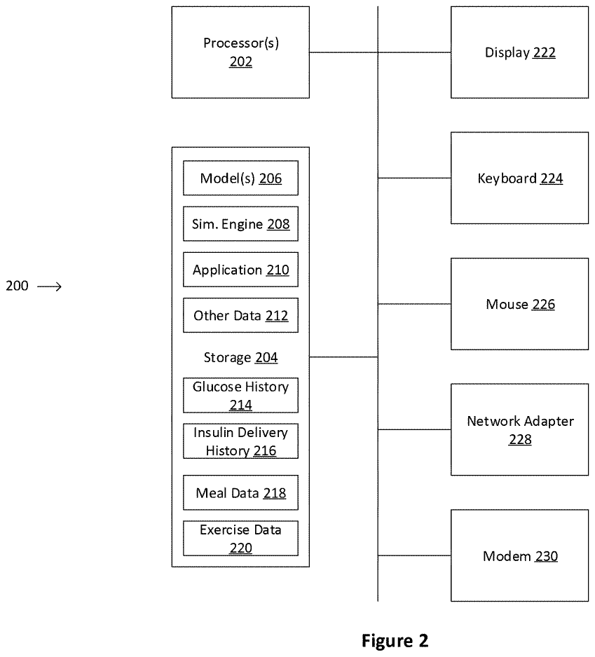

As mentioned above, the exemplary embodiments may perform simulations of the effects a change in control method settings may have on glycemic outcomes of the user 110. The simulations and related activities may be performed by a computing system 200 like that depicted in FIG. 2. The computing system 200 may include one or more processors 202. The processor(s) 202 may take many forms, such as discussed above relative to processor 104. The processor(s) 202 may perform simulations such as described below. The computing system 200 may contain a storage 204 for storing computing programming code, data, documents and other types of files. The storage 204 may include a variety of different storage components, such as discussed above relative to storage 106. The storage 204 may store one or more models 206 of a medicament delivery device. These model(s) 206 may be simulated by a simulation engine 208. The simulation engine 208 may be stored in the storage 204 and run by the processor(s) 202. The simulation engine 208 may be, for example, a simulation engine provided by the MATLAB technical computing environment, a simulation engine provided by the Arena simulation environment, a simulation engine provided by Simio, etc. The storage 204 may store an application 210 that obtains the simulation results and chooses the best weights and in silico user as described herein. The application 210 may provide a user interface for displaying and comparing simulation results and may compute performance metrics like TIR and time below range (TBR). The application 210 may be run by the processor(s) 202.

The storage 204 may store data such as the glucose history 214 of the user and possibly other users. The storage 204 may store the insulin delivery history of the user. More generally, the storage may store an analyte history of the user of one or more analytes and may store the medicament delivery history of the user. The storage may store meal data 218 for meals ingested by the user and may store exercise data 220 for the user that may specify type of exercise, duration of exercise, intensity of exercise, etc. The storage may also store other data 212.

The computing system 200 may include a display 222, such as a liquid crystal display (LCD), a light emitting diode (LED) display, a touchscreen display, a retinal display, or the like. A keyboard 224 and a mouse 226 may be provided for entering input. A network adapter 228 may be provided for connecting with a network, such as a local area network, like a WiFI network and/or an Ethernet network, or a wide area network. A modem 230, such as a telephone modem or a cable modem may be provided.

The computing system 200 may be or may include a computing device like a laptop computer, a desktop computer, a server computer, a tablet computer, a smartphone, or other type of computing device. The components of FIG. 2 may be part of such a computing device.

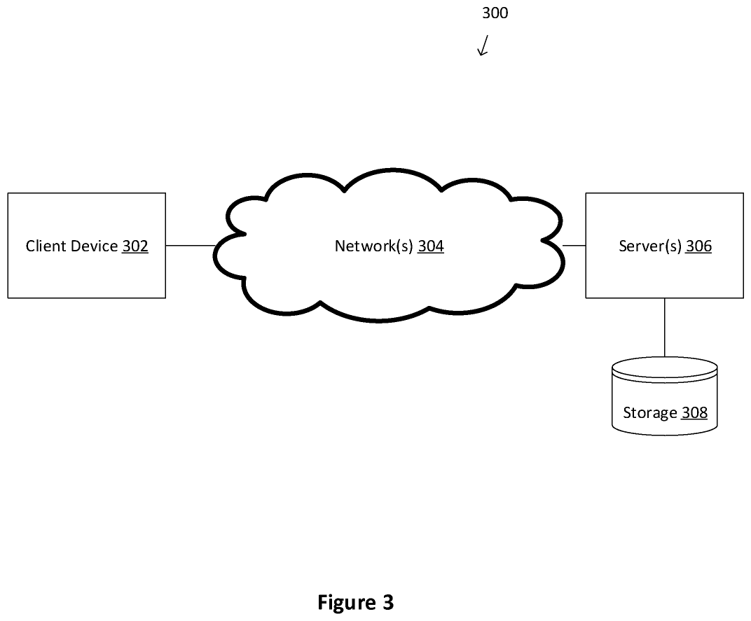

The simulations may be performed by a tightly coupled system like that shown in FIG. 2 or may be performed in a distributed computing environment 300 like that shown in FIG. 3. The term “computing environment” refers to its common meaning in the art. In particular it may refer to the combination of hardware, software, network resources, and services required to run and manage applications, ranging from standalone computers to distributed cloud systems. The distributed computing environment 300 includes a client device 302 coupled to one or more networks 304. Client device 302 may be a computing system like computing system 200 shown in FIG. 2. The network(s) 304, may include both local area networks (LANs) and wide area networks (WANs), like the Internet. The client device 302 may request service from one or more server(s) 306. The server(s) may be realized as one or more computing devices. In some cases, the server(s) 306 may be cloud resources. A storage 308 may be available to the server(s). In the exemplary embodiments, the client device 302 may request simulations of one or models be performed by the server(s) 306, and the results of the simulations be sent over the network(s) to the client device 302. The server(s) 306 may include the components like those shown in FIG. 2. For example, the server(s) 306 may include the data and computing programming instructions shown in storage 204 in some embodiments and may include processor(s) for executing the computer programming instructions.

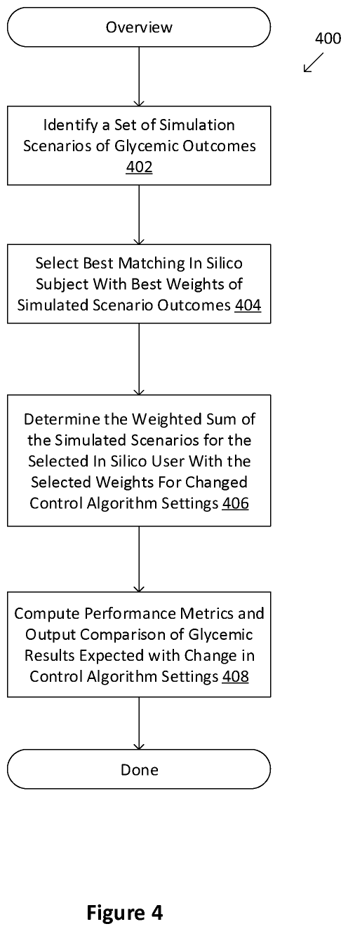

FIG. 4 depicts a flowchart 400 of illustrative steps that may be performed in exemplary embodiments. At 402, a set of simulation scenarios for glycemic outcomes are identified for use. The exemplary embodiments recognize that a single normal simulation scenario may not accurately capture user behavior and user characteristics that affect glucose levels over the course of a day. Thus, the exemplary embodiments may use a plurality of simulation scenarios in combination to more accurately simulate anticipated user behavior and user characteristics for a user. Each scenario is constructed by establishing simulation parameters that will capture the scenario. For example, an input for the simulation may be meal size. One scenario may require a large meal size, whereas another scenario may require a small meal size. Similarly, meal composition may be captured by simulation parameters that may vary among scenarios. The term “simulation scenario of glycemic outcomes” as used herein refers to its common meaning in the art. In particular it may refer to a defined set of parameters used as inputs to a simulation of a user's glycemic response, wherein different scenarios correspond to different combinations of parameters such as food intake and/or physical activity, for simulating corresponding glycemic outcomes. The term “parameter” refers to a variable input, having a discrete or continuous value, that represents a user-related, physiological, behavioral, or treatment-related factor and is used in defining a simulation scenario for generating glycemic outcomes.

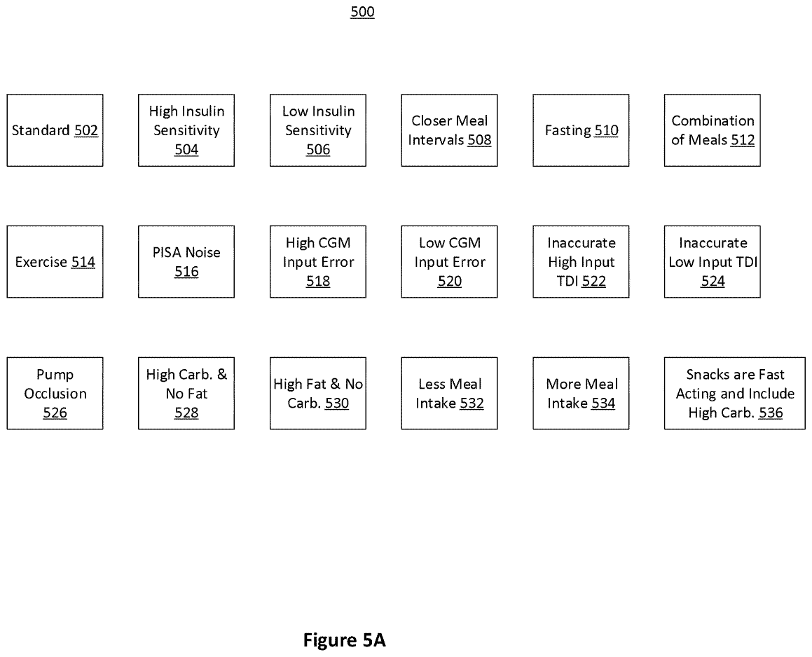

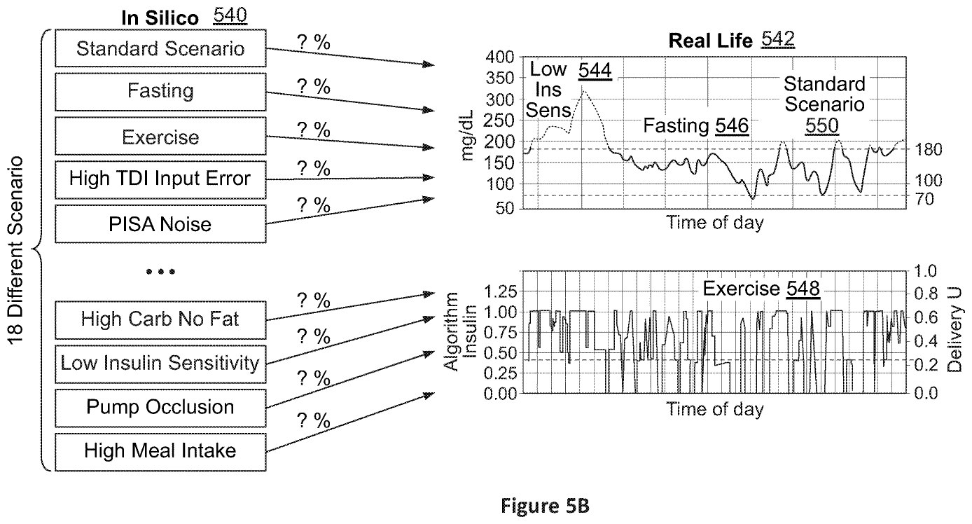

FIG. 5A depicts examples of simulation scenarios 500 that may be used in the exemplary embodiments. The standard simulation scenario 502 may represent typical behavior of the user with typical user characteristics. The high insulin sensitivity scenario 504 may represent the case where the user has a high insulin sensitivity and thus the glucose level of the user reacts more strongly than normal to insulin delivery. The low insulin sensitivity simulation scenario 506 may represent the case where the user has a low insulin sensitivity and thus the glucose level of the user reacts less strongly than normal to insulin delivery. The “closer meal intervals” simulation scenario 508 is one in which the user consumes meals or snacks closer in time than normal. The fasting simulation scenario 510 represents when a user fasts. In the combination of meals simulation scenario 512, multiple meals are consumed together (e.g., breakfast and lunch are consumed as one meal). The term “user fasting” as used herein refers to its common meaning in the art. In particular it may refer to a metabolic state in which no caloric intake, in particular no carbohydrate intake, has occurred for a predefined period of time sufficient for the user's glucose dynamics to be substantially independent of recent meal ingestion. The predefined period of time may correspond to a time interval following a detected or reported meal, optionally on the order of one to several hours, for example at least 1 hour, at least 2 hours, or at least 3 hours after the meal. Alternatively or additionally, the onset of the fasting state may be determined based on one or more physiological indicators, including a stabilization of glucose levels, a rate of change of glucose falling below a predefined threshold, an absence of a postprandial glucose rise, or a model-based determination that exogenous glucose absorption from a prior meal has substantially ceased. In some embodiments, the start of the predefined period is determined based on a detected meal event, a user-reported meal, or an inferred meal based on glucose dynamics. The terms “high insulin sensitivity” and “low insulin sensitivity” as used herein refer to their common meaning in the art. In particular a “high insulin sensitivity” may refer to a condition in which an insulin sensitivity factor, defined as a decrease in blood glucose per unit of administered insulin, exceeds a predefined upper threshold value, and the term “low insulin sensitivity” may refer to a condition in which the insulin sensitivity factor falls below a predefined lower threshold value. For example, low insulin sensitivity may be defined as an insulin sensitivity factor falling within a lower quantile of a distribution of insulin sensitivity values, and high insulin sensitivity may be defined as an insulin sensitivity factor falling within an upper quantile of the distribution. Other percentile ranges may be selected to define “low insulin sensitivity” and “high insulin sensitivity”.

The exercise simulation scenario 514 is one in which the user exercises. The pressure induced sensor (PISA) noise simulation scenario 516 simulates when there is PISA noise, such as when a user induces pressure on a glucose sensor by sleeping or sitting on top of the sensor. The high continuous glucose monitor (CGM) input error simulation scenario 518 represents when the glucose sensor values used by the control system have an error that makes them too high. The low CGM input error simulation scenario 520 represents when the glucose sensor values used by the control system have an error that makes them too low. The inaccurate high input TDI simulation scenario 522 represents when the total daily insulin (TDI) value that is input is too high for the user. The inaccurate low input TDI simulation scenario 524 represents when the TDI value that is input is too low for the user. The term “total daily insulin inaccuracy” as used herein refers to its common meaning in the art. In particular it may refer to a condition in which a TDI, defined as a control variable used by a system as a control system setting representing a total amount of insulin to be delivered over a predefined daily period, deviates by more than a predefined threshold value from a corresponding reference TDI representing an actual insulin requirement of the user over the predefined daily period. The total daily insulin inaccuracy may also be expressed as a numerical value corresponding to an amount of the deviation.

The pump occlusion simulation scenario 526 represents when the pump delivering medicament is occluded. The high carbohydrates and no/low fat simulation scenario 528 is when the ingested meal(s) are high in carbohydrates and low in fat. The high fat and no/low carbohydrates simulation scenario 530 is when the ingested meal(s) are high in fats and low in carbohydrates. The less meal intake simulation scenario 532 represents when the user intakes less in meals than normal. The more meal intake simulation scenario 534 represents when the user intakes more in meals than normal. The snacks are fast acting and include high carbohydrates simulation scenario 536 represents when the user ingest snacks that raise glucose level quickly and contain a high amount of carbohydrates. The term “high carbohydrate ingestion” and “low carbohydrate ingestion” as used herein refer to their common meaning in the art. In particular “high carbohydrate ingestion” may refer to a condition in which an amount of ingested carbohydrates within a predefined time interval exceeds a predefined upper threshold value, and the term “low carbohydrate ingestion” refers to a condition in which the amount of ingested carbohydrates within the predefined time interval falls below a predefined lower threshold value. For example, low carbohydrate ingestion may be defined as an amount of ingested carbohydrates falling within a lower quantile of a distribution of carbohydrate intake values, and high carbohydrate ingestion may be defined as an amount of ingested carbohydrates falling within an upper quantile of the distribution. Other percentile ranges may be selected.

The simulation scenarios may be constructed by varying simulation parameters. For example, meal size in an input parameter that may be adjusted in the simulation model(s). The composition of meals may also be adjusted by way of the input parameters. For example, parameters may vary the amount of fat and carbohydrates in meals. Other simulation parameters may specify insulin sensitivity, the timing of meals, and omission of meals.

In some exemplary embodiments, all of the simulation scenarios may be used. In other embodiments, a subset of these scenarios may be used. It should be appreciated that simulation scenarios other than those depicted in FIG. 5A may be used. These simulation scenarios are intended to be illustrative and not limiting.

As depicted in FIG. 5B, the challenge is figuring out how to combine the in silico scenarios 540 to produce a result that accurately reflects the real life glucose levels 542 of the user. For example, the user may have low insulin sensitivity, may fast at times, may exercise at times, and may behave in a manner consistent with standard expected behavior. Thus, the real life glucose levels may reflect a combination of multiple in silico scenarios. In the depicted example, the real life glucose levels are captured by a combination of at least the low insulin scenario 544, a fasting scenario 546, an exercise scenario 548, and a standard scenario 550. The exemplary embodiments determine percentages to be applied to each of the in silico scenarios to best capture the user's real life behavior and glucose levels.

At 404, a best in silico subject with best weights for the simulation scenarios are selected. The simulations rely on in silico subjects. Each in silico subject may have a different physiology relative to other subjects. For example, subjects may vary as to age, sex, weight, and other physiological characteristics. Each in silico subject may, in some instances represent a subject in a clinical trial. When a simulation scenario is run by the simulation engine 208, it is run for a particular in silico subject. As is explained in more detail below, each of the simulation scenarios may be run for each of the in silico subject by the simulation engine 208. Based on simulation results for the scenarios, the best in silico subject and best weights are selected.

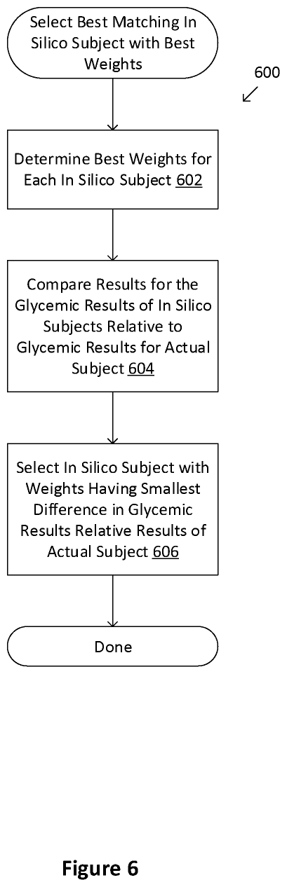

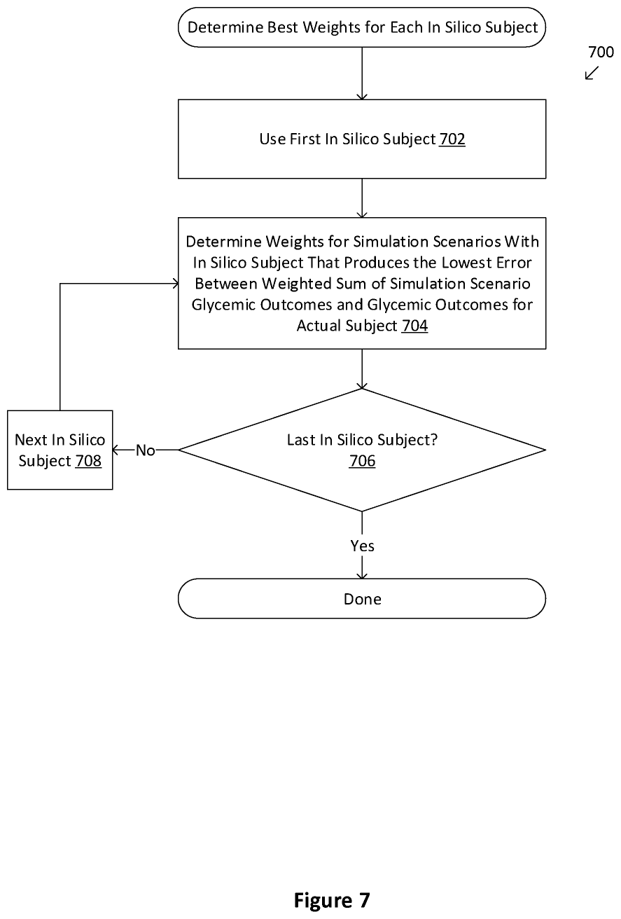

FIG. 6 depicts a flowchart 600 of illustrative steps that may be performed in exemplary embodiments to select the best matching in silico subject and best matching weights for a user (i.e., step 404). At 602, the best weights for each in silico subject are determined. FIG. 7 depicts in more detail how the best weights for each in silico subject may be determined in exemplary embodiments. At 702, a first in silico subject is used to produce simulation results for each of the simulation scenarios given current control parameter settings for the current insulin therapy. In other words, a separate simulation is run for each simulation scenario where the subject is the first in silico subject. The simulation results specify a specific glycemic outcome (i.e., specific glucose level values for the subject at times in a time window). At 704, the weights that produce the lowest error between a weighted sum of the glycemic outcomes for each of the simulation scenarios and the actual glycemic outcome for the user are determined based on the glucose history. Hence, weights are chosen to produce resulting glucose values for the times in the time period, where each resulting glucose value is a weighted sum of the glucose level values of the simulation scenarios for a specific time in the time period. The weights are chosen so that the resulting glucose level values from the weighted sums over times in the time window have a lowest error (as measured by an objective function) relative to the actual glycemic outcome of the user (i.e., the actual glucose level values of the user for the times in the time window). An optimization methodology, such as fmincon optimization method available in MATLAB may be used to find the best weights. This methodology may be an iterative process where at least one weight is adjusted for each iteration.

The error may be specified by the objective function:

E=(TI R_{actual}−TI R_{weighted_~_outcome})·3+(Hypo_{actual}−Hypo_{weighted_~_outcome}·10)

where E is the error, TIR_{actual} is the time in range (e.g., between 120 mg/dL and 70 mg/dL) for the actual glycemic outcome of the user, TI R_{weighted_~_outcome} is the time in range for the weighted sum of the glycemic outcomes for the simulation scenarios, Hypoactual is the number of hypoglycemic events (e.g., glucose level below 70 mg/dL) that the user actually experienced, and Hypo_{weighted_~_outcome} is the number of hypoglycemic events experienced in the simulation glycemic outcomes calculated using the weighted sum of the results from the simulation scenarios. The parameters of this objective function, such as 3 and 10, are variable and may be tuned based on the relative weights to be applied to the accuracy of prediction of hypoglycemia versus the Time in Range values.

After the best weights are determined for the first in silico subject, the next in silico subject is chosen at 708. In some cases, only one in silico subject may be simulated so the process is not repeated for other in silico subjects. The process is repeated at 704 until the best weights for all of the in silico subjects are processed (see 706).

At 604, once the best weights are determined for the glycemic results of the simulation scenarios in 602, the weighted sum of the glycemic outcomes for simulation scenarios for each in silico subject are compared to the actual glycemic outcome for the user. At 606, the in silico subject and weights that results in the least error may be selected.

At 406, at least one of the control method settings may be modified, and the weighted sum of the glycemic outcomes produced for the selected in silico subject with the selected weights may be calculated. At 408, performance metrics, such as percentage of TIR and percentage of TBR may be determined. A comparison of the of the weighted sum of the glycemic outcomes for the simulation scenarios with the selected weights (which is a best estimate of how the glycemic outcome of the user will change due to the change in the one or more control method settings) and the glycemic outcome of the user prior to the change may be output (e.g., to the user). The performance metrics for the simulated glycemic outcome and the actual glycemic outcome may be output instead or in addition to the glycemic outcome values. The output may be shown on the display 222 and may include one or more of text, graphics, and video content. The comparison helps in understanding how the change in the one or more control method settings will affect the glycemic outcome of the user. The output may include one or more performance metrics, such as TIR, TBR, time above range (TAR), mean glucose, glucose management indicator (GMI), coefficient of variation (CV) of glucose, standard deviation of glucose, mean amplitude of glycemic excursions (MAGE), continuous overall net glycemic action (CONGA), low blood glucose index (LBGI), high blood glucose index (HBGI), glycemic risk index (GRI), area under the curve (AUC) below a threshold, area under the curve above a threshold, number and/or duration of hypoglycemic events, postprandial peak glucose, time to return to range, and/or overnight time in range. In particular the output may include TIR, TBR, TAR, and CV. One or more of these metrics may be combined to form a composite score indicative of the quality of glucose control which may additionally or alternatively be output. The output may additionally or alternatively comprise a recommendation to the user, whether to implement the change of the at least one control system setting. The output may also include generating a user interface element on a display, the user interface element being selectable to apply and/or decline the change in control method settings. Alternatively, the change in control method settings may also be applied automatically, in particular if the improvement in performance metrics exceeds a predefined threshold.

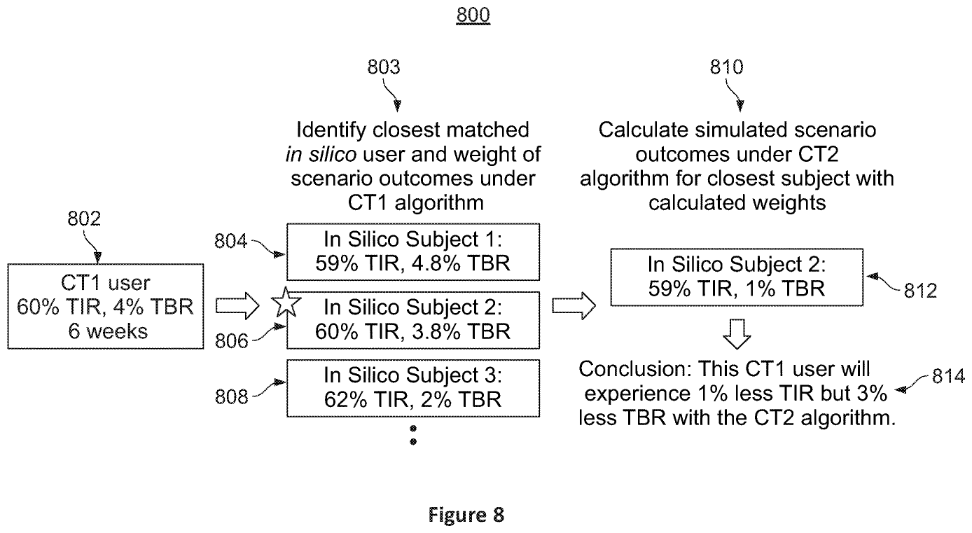

FIG. 8 provides a graphic representation of the process described above. Suppose that a user's current glycemic outcome over a six week period is 60% TIR and 4% TBR (see 802). At 803, the closest matched in silico user and weights are identified. As can be seen in 806, the weighted sum of glycemic outcomes in silico subject 2 better matches the glycemic outcome 802 of the actual user than the weighted sums of the glycemic outcomes 804 and 808 of in silico subjects 1 and 3, respectively. Thus, at 810, the weighted sum 812 of the glycemic outcomes for the simulation scenarios for in silico subject 2 are calculated with the control method setting changes. At 814, the results of the weighted sum 812 are compared to the results 802 of the actual subject to note that the user will experience 1% less TIR and 3% less TBR.

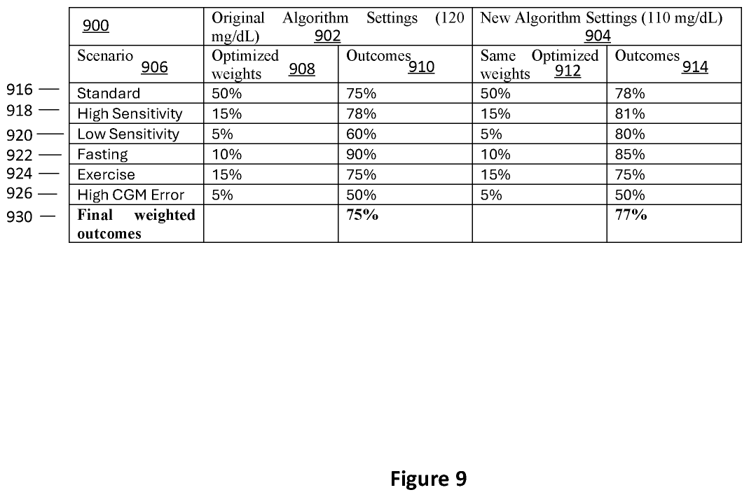

FIG. 9 depicts an illustrative table 900 that compares glycemic outcomes. This table 900 or an equivalent table may be output on the display 222 in some embodiments. The table compares original method settings 902 with new method settings 904, where the new method settings are those that result from changing one or more of the control method settings. Each row of the table 916-926 represents data for an associated simulation scenario. For example, row 916, holds data for the standard simulation scenario. The data includes optimized weights 908 for the simulation scenarios and outcomes 910 that are specified as TIR percentage. Hence, for the standard simulation scenario, the optimized weight is 50% or 0.5 and the TIR percentage is 75%. When the control method settings are changed to the new method settings 904, the weight 912 of standard scenario stays at 50% but the percentage TIR 914 changes to 78%. There is a final row 930 in the table 900 that sums the total percentage TIR across the weighted simulation scenarios. As can be seen, in this example, the cumulative percentage TIR for the original control method settings is 75%, whereas the cumulative percentage for the new adjusted control method settings is 77%. This is a 2% increase in percentage TIR. Such an increase in TIR, though it may appear to be small, represents a tremendous positive impact on the life of a person living with diabetes.

While exemplary embodiments have been described herein, various changes in form and detail relative to the exemplary embodiments may be made without departing from the intended scope of the claims appended hereto and equivalents thereof.

1. A method performed by one or more processors of a computing environment, comprising:
determining with the one or more processors a weighted mix of simulation scenarios of glycemic outcomes for a user of an insulin delivery device, wherein the weighted mix specifies weights for results for each of the simulation scenarios; and
determining with the one or more processors an estimated glycemic outcome for the user given a change of at least one control system setting of the insulin delivery device by determining results of simulating with the one or more processors each of scenarios in the weighted mix of simulation scenarios with the changed at least one control system setting and weighing the results per weights specified in the weighted mix of simulation scenarios.

2. The method of claim 1, wherein the determining of the weighted mix of simulation scenarios is based on a glucose level history of the user.

3. The method of claim 2, wherein the weights are determined by applying an optimization to yield weights that produce a weighted sum of scenario results that is similar to actual glucose levels of the user in the glucose level history of the user.

4. The method of claim 1, wherein the scenarios include at least one of a scenario for high insulin sensitivity, a scenario for low insulin sensitivity, a scenario for user fasting, or a scenario for user exercising.

5. The method of claim 1, wherein the scenarios include at least one scenario for pump occlusion, a scenario for pressure induced sensor error, a scenario for total daily insulin inaccuracy, or a scenario for glucose sensor error.

6. The method of claim 1, wherein the scenarios include at least one scenario for high carbohydrate ingestion, a scenario for high fat ingestion, a scenario for ingesting multiple meals, or a scenario for eating meals closer in time than normal.

7. The method of claim 1, further comprising outputting information regarding a difference between the estimated glycemic outcome for the user given the change of the at least one control system setting versus an actual glycemic outcome for the user without the change of the at least one control system setting.

8. A non-transitory computer-readable storage medium storing computer programming instructions that when executed by a processor cause the processor to:
determine a weighted mix of simulation scenarios of glycemic outcomes for a user of an insulin delivery device, wherein the weighted mix specifies weights for results for each of the simulation scenarios; and
determine an estimated glycemic outcome for the user given a change of at least one control system setting of the insulin delivery device by determining results of simulating each of scenarios in the weighted mix of simulation scenarios with the changed at least one control system setting and weighing the results per weights specified in the weighted mix of simulation scenarios.

9. The non-transitory computer-readable storage medium of claim 8, wherein the determining of the weighted mix of simulation scenarios is based on a glucose level history of the user.

10. The non-transitory computer-readable storage medium of claim 9, wherein the weights are determined by applying an optimization to yield weights that produce a weighted sum of scenario results that is similar to actual glucose levels of the user in the glucose level history of the user.

11. The non-transitory computer-readable storage medium of claim 8, wherein the scenarios include at least one of a scenario for high insulin sensitivity, a scenario for low insulin sensitivity, a scenario for the user fasting, or a scenario for the user exercising.

12. The non-transitory computer-readable storage medium of claim 8, wherein the scenarios include at least one scenario for pump occlusion, a scenario for pressure induced sensor error, a scenario for total daily insulin inaccuracy, or a scenario for glucose sensor error.

13. The non-transitory computer-readable storage medium of claim 8, wherein the scenarios include at least one scenario for high carbohydrate ingestion, a scenario for high fat ingestion, a scenario for ingesting multiple meals, or a scenario for eating meals closer in time than normal.

14. The non-transitory computer-readable storage medium of claim 8, wherein the computer programming instructions, when executed by the processor, further cause the processor to output information regarding a difference between the estimated glycemic outcome for the user given the change of the at least one control system setting versus an actual glycemic outcome for the user without the change of the at least one control system setting.

15. A method performed by a processor of a computing system, comprising:
with the processor simulating glycemic outcomes of different in silico users of an insulin delivery device for different scenarios using an initial set of control system settings, the in silico users differing as to physiology;
with the processor, determining weights of the glycemic outcomes of the scenarios for each in silico user to produce a weighted sum of the glycemic outcomes for the scenarios that most closely matches an actual glycemic outcome for the in silico user of the insulin delivery device given the initial set of control system settings;
with the processor, selecting a weighted sum of the glycemic outcomes for the scenarios for a selected one of the in silico users that most closely matches the actual glycemic outcome for the in silico user of the insulin delivery device;
with the processor, simulating the scenarios for the selected one of the in silico users given a new set of control system settings to determine an updated weighted sum of the glycemic outcomes for the scenarios for the selected in silico user as an estimate of a glycemic outcome for a user of the insulin delivery device; and
outputting information regarding the updated weighted sum.

16. The method of claim 15, further comprising outputting information regarding the actual glycemic outcome for the user of the insulin delivery device given the initial set of control system settings.

17. The method of claim 15, wherein the determining of the weights is based on glucose levels in a glucose level history of the user.

18. The method of claim 17, wherein the weights are determined by applying an optimization to yield weights that produce a weighted sum of the glycemic outcomes for the scenarios that is similar to actual glucose levels of the user in the glucose level history of the user.

19. The method of claim 15, wherein the insulin delivery device is an insulin patch pump.

20. The method of claim 15, further comprising determining performance metric values for both the actual glycemic outcomes for the user of the insulin delivery device with the initial set of control system settings and the estimate of the glycemic outcome for the user of the insulin delivery device with the new set of control system settings.
